(19) United States
(12) Patent Application Publication
(10) Pub. No.: US 2008/0199351 A1
(43) Pub. Date: Aug. 21, 2008

(54) ZERO YIELD REACTOR AND METHOD OF SANITIZING AIR USING ZERO YIELD REACTOR

(75) Inventor: Terrence O. Woodbridge, Cary, NC (US)

Correspondence Address: MCGINN INTELLECTUAL PROPERTY LAW GROUP, PLLC, 8321 OLD COURTHOUSE ROAD, SUITE 200, VIENNA, VA, 22182-3817, US

(73) Assignee: AirOCare, Inc., Rockville, MD (US)

(21) Appl. No.: 12/032,398

(22) Filed: Feb. 15, 2008

Related U.S. Application Data
(60) Provisional application No. 60/901,346, filed on Feb. 15, 2007.

(51) Int. Cl. A61L 9/015

(52) U.S. Cl. 422/4; 422/120; 422/122

(57) ABSTRACT

An apparatus (and method) for sanitizing air includes a generating unit that generates reactive oxygen species and ozone from oxygen in air, received by the generating unit, to be sanitized, and a cancellation unit or conversion filter that cancels at least a portion of the ozone before the air is returned to an area to be sanitized.

### CROSS REFERENCE TO RELATED APPLICATIONS

The present application relates to U.S. provisional Patent Application No. 60/901,346, to Terrence Woodbridge, entitled “ZERO YIELD REACTOR”, which is incorporated, in its entirety, herein by reference.

### BACKGROUND OF THE INVENTION

1. Field of the Invention

The present invention relates to a method and apparatus for sanitizing air (environment), surfaces and spaces through the generation and cancellation of reactive oxygen species.

2. Description of the Related Art

Temperature changes, changes in the moisture in the air feeding into heating, ventilation, and air-conditioning (HVAC) systems increase the number of micro-organisms in the air, producing increased colonies of certain fungi and bacteria, both of which are potentially harmful.

HVAC systems in office buildings, as well as hospitals, can be a source of various pathogens, which spread infectious micro-organisms from one zone to another. This is the principal cause of Sick Building Syndrome, recognized by the World Health Organization as a threat to healthy work and living environments.

The purification of environments can be achieved through the use of ozone. Ozone has been used to purify air conditioning systems in buildings and to sanitize warehouses where products are stored. Despite its widespread use, this basic technique has a disadvantage of accumulating more ozone than is desirable in the treated environment, requiring the elimination of the excess ozone. Several different improvements in this method have been made in an attempt to control the levels of ozone in the environment being treated.

One such improvement provides high initial levels of ozone to the environment sufficient to produce the desired bacteriostatic or bactericidal effect. Later, the levels of ozone are reduced so that they do not produce harmful effects to the products being treated or to humans in the environment.

However, the majority of the known systems for purifying closed areas with ozone are based on an ozone generator that utilizes a source of concentrated oxygen, for example bottled oxygen or a known pressurized oxygen generating system utilizing static discharge. When ozone is generated from a source of concentrated oxygen, the level of oxygen in the enclosure may rise along with the level of ozone. The increase in oxygen levels is due to the breakdown of ozone partially into new molecules of oxygen. An increase in the level of oxygen in enclosures containing natural perishable products enhances cellular metabolism and thus is detrimental to the storage of the perishable products.

One known method is applied to substantially closed rooms or rooms with a controlled atmosphere. The substantially closed room includes a closed circuit air conditioning system, such as a cooling system, for the preservation of perishable natural products. A known ozone generator is placed in proximity with the substantially closed room such that the ozone generator can draw in air from within the substantially closed room and liberate ozone into the substantially closed room. In contrast to other known ozonation systems, the known method utilizes oxygen from the air of the room in which the purification treatment is being applied to generate ozone. Because the method converts oxygen from the air into ozone, no increase in oxygen levels is observed in the closed room. Rather, the gaseous equilibrium is shifted so that there is maintenance of the level of oxygen in the enclosure due to the reversion of ozone into oxygen after a short period of time.

The oxidative character of the ozone has a bacteriostatic and fungistatic effect in the short term, followed by a bactericidal and fungicidal effect. These effects combine with the lowered metabolism in a temperature cooled environment to reduce ripening, retard spoilage and thus preserve natural perishable products stored in the room.

However, the system does not provide an optimal means for efficiently sanitizing the air within the closed room.

Furthermore, the conventional systems introduce a high level of ozone into the environment.

Additionally, the conventional system cannot produce reactive oxygen species and ozone, while eliminating the ozone before it is expelled into the room air.

Finally, the conventional systems produce nitric acid (the combination of a high level of ozone and humidity).

### SUMMARY OF THE INVENTION

While ozone may be used to purify air in a hospital by removing bacteria and viruses related to (but not limited to) staph infections, mRSA, norovirus, influenza, etc., the presence of ozone in a surgical environment or ill patient area is not desirable. Indeed, hospital regulations require that no device produce more than 0.05 parts per million (ppm) of ozone. This includes light fixtures, electric motors, copiers, etc. Accordingly, it is desirable to provide an air sanitizing method that does not emit ozone into the environment being sanitized.

In view of the foregoing and other exemplary problems, drawbacks, and disadvantages of the conventional methods and structures, an exemplary feature of the present invention is to provide a method and apparatus generating reactive oxygen species and ozone, treating the air to be sanitized with the generated reactive oxygen species including ozone, and canceling the ozone, while maintaining the other reactive oxygen species, to efficiently sanitize the air and surfaces with little or zero yield of ozone.

In a first exemplary, non-limiting aspect of the present invention, an apparatus for sanitizing air includes a generating unit that generates reactive oxygen species including ozone from oxygen in air, received by the generating unit, to be sanitized, and a cancellation unit that cancels at least a portion of the ozone before the air is returned to an area to be sanitized.

In a second exemplary, non-limiting aspect of the present invention, an apparatus for sanitizing air includes a reactor including a plurality of arrays. The plurality of arrays include a reactive species generating array that generates reactive oxygen species including ozone from oxygen in the air, received by the generating unit, to be sanitized, and a cancellation array that cancels at least a portion of the ozone before the air is returned to an area to be sanitized.

In a third exemplary, non-limiting aspect of the present invention, an apparatus for sanitizing air includes a reaction unit that generates reactive oxygen species including ozone from oxygen in air, received in the reaction unit, to be sanitized, and a conversion filter that converts the ozone back to oxygen.

In a fourth exemplary, non-limiting aspect of the present invention, a method of sanitizing air includes generating reactive oxygen species including ozone from oxygen in air to be sanitized and canceling at least a portion of the ozone before the air is returned to an area to be sanitized.

In a fifth exemplary, non-limiting aspect of the present invention, a method of sanitizing air includes generating reactive oxygen species including ozone from oxygen in air to be sanitized, and canceling at least a portion of the ozone before the air is returned to an area to be sanitized by applying a space cancellation differential.

In a sixth exemplary, non-limiting aspect of the present invention, a method of sanitizing air includes generating reactive oxygen species including ozone from oxygen in air to be sanitized and converting the ozone back to oxygen or other reactive oxygen species.

Accordingly, the apparatus (and method) of the present invention is designed and calibrated to be capable of producing little to no ozone, while still being capable of producing other reactive oxygen species. Through the process of cancellation, the apparatus (and method) of the present invention reduces the production level of ozone to a point that there are substantially no traceable levels of ozone within the treated environment.

The apparatus of the present invention is capable of producing reactive oxygen species while limiting and/or eliminating the amount of ozone introduced into the environment. This provides the ability to turn on and turn off the cancellation or conversion of ozone so that during certain selected periods (e.g., when an operating room is not occupied) the ozone and other reactive oxygen species can be emitted to provide additional sanitation.

The apparatus (and method) of the present invention may be used in a room, a duct system, and to sanitize heating and cooling ducts and equipment.

Furthermore, the apparatus (and method) of the present invention is capable of reducing or eliminating ozone from an environment. For example, an aircraft flying at an altitude has a high concentration of ozone in the cabin air from air intake at that altitude.

Additionally, the apparatus (and method) of the present invention is capable of treating contaminated air being expelled from a space with no ozone emissions. For example, air from an infectious disease portion of the hospital or air containing other contaminants including odors.

Moreover, the apparatus (and method) of the present invention is useful in treating and decontaminating air that is moving from one space to another. For example, the creation of a sanitized air curtain for use in a biotechnology laboratory between office space and so-called clean rooms.

These and many other advantages may be achieved with the present invention.

### BRIEF DESCRIPTION OF THE DRAWINGS

The foregoing and other exemplary purposes, aspects and advantages will be better understood from the following detailed description of an exemplary embodiment of the invention with reference to the drawings, in which:

FIG. 1 illustrates a reactor 100 according to an exemplary, non-limiting embodiment of the present invention;

FIG. 2A illustrates a zero yield reactor 200 according to an exemplary, non-limiting embodiment of the present invention;

FIG. 2B illustrates a filter 209 in accordance with the exemplary, non-limiting embodiment illustrated in FIG. 2A; and

FIG. 3 illustrates a zero yield reactor 300 according to an exemplary, non-limiting embodiment of the present invention.

### DETAILED DESCRIPTION OF EXEMPLARY EMBODIMENTS OF THE INVENTION

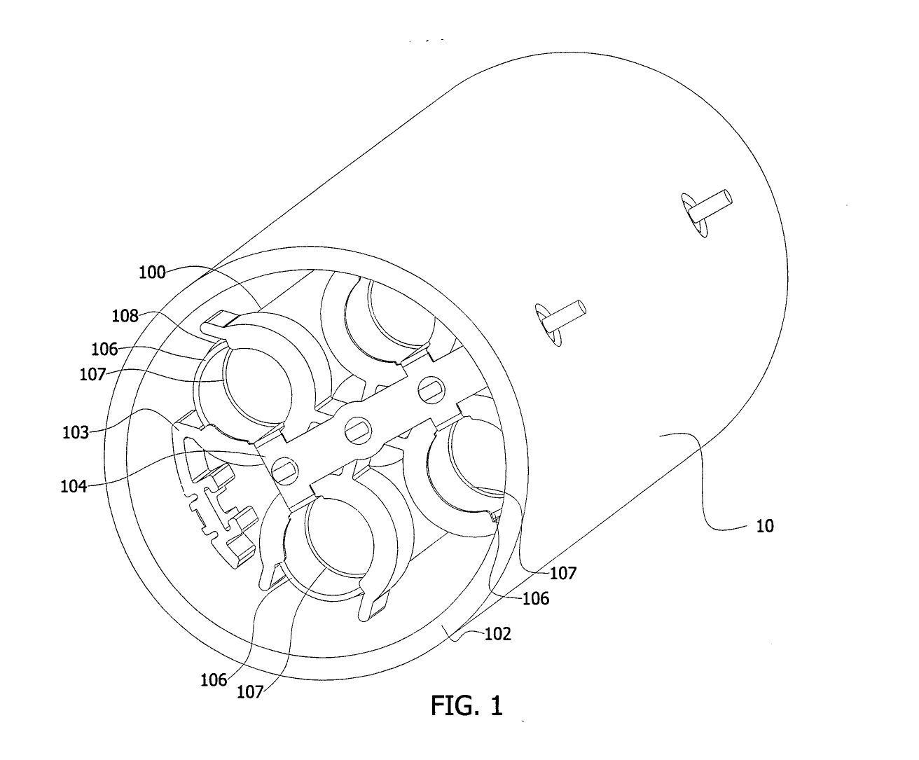

Referring now to the drawings, and more particularly to FIGS. 1-3, there are shown exemplary embodiments of the method and structures of the present invention.

In accordance with certain exemplary embodiments of the present invention, the zero yield reactor includes a reactor that is designed and calibrated to produce very little (e.g., approximately 0.02 ppm) ozone (O₃), while still having the ability to produce other reactive oxygen species. Through a process of cancellation, the present apparatus (and method) may reduce the production level of ozone so that, if appropriate, substantially no traceable levels of ozone are present within a treated environment.

The reactor produces (e.g., constantly produces) reactive oxygen species (ROS), which, for purposes of this patent application, include ozone and other reactive oxygen species, through the generation of a corona. A corona is a process by which a current develops from an electrode with a high potential in a neutral fluid, usually by air, by ionizing that fluid to create plasma around an electrode. The ions generated eventually pass charge to nearby areas of lower potential, or recombine to form neutral gas molecules.

In accordance with certain exemplary embodiments, the zero yield reactor of the present invention may use one or more of the following ozone cancellation mechanisms.

First, by pulsating, at a very fast rate (e.g., 100 ns), a voltage within a range of 2000 VAC to 5000 VAC (more preferably at a voltage of approximately 3,500 volts AC) to the reactor cell, the zero yield reactor is able to control pooling of the plasma and reduce the corona region (e.g., reduced to 96 mm-192 mm).

Second, by generating very low frequencies (e.g., 12 kHz) to the reactor cell, the plasma field can be increased without allowing the field to collapse. Accordingly, the production of ozone is reduced.

Third, controlling the size of an air gap between the mesh and the glass (described in detail below) helps with airflow and cooling of the reactor. The size of the mesh is also important to the dielectric (e.g., glass tube or ceramic) because this sets the air space between the dielectric and the steel mesh. The length (i.e., a preferred length may be 85% coverage of the dielectric) and wire gauge (e.g., 18) dictate the size of the corona field.

Fourth, control over sharp edges within the steel mesh and contacts of the reactor control pooling and electrical discharge.

Fifth, the quality, thickness, and makeup of the dielectric (described in detail below) have an impact on the production level and overall quality of the plasma produced. A very high grade of glass (e.g., Duran borosilicate) yields the best results, but also has a lower ability to dissipate heat.

Finally, by placing multiple cells in front of one another at a distance in a range of 20 mm to 25 mm (more preferably at a distance of approximately two inches) allows the zero yield reactor to cancel any remaining ozone. Preferably, the spacing between each of the cells is the same. If the spacing becomes larger or smaller downstream of the air flow, then the cancellation effect is decreased.

FIG. 1 illustrates a reactor 10 in accordance with exemplary aspects of the present invention. The reaction unit generates reactive oxygen species, including ozone, from oxygen (O₂) in the air received through an intake port.

The air received through the intake port is preferably ambient air from the environment that is to be treated. The introduction of air into the reactor 10 may be mediated through a forced suction or by natural suction. When mediated through a forced suction, the zero yield reactor apparatus may contain a turbine, which draws air into the reactor 10 through the intake port. Preferably, the air is drawn through a filter to remove dust and other macroscopic impurities that may be present in the air to be sanitized before the air enters the reactor 10.

The reactor 10 splits the oxygen in the air into large amounts of reactive oxygen species. The reactive oxygen species generated may include singlet oxygen (1O₂), ozone (O₃), atomic oxygen (O), superoxide (O₂—), hydrogen peroxide (H₂O₂), hydroxyl radical (OH—), and peroxynitrite (ONOO—), among others. Even though many reactive oxygen species have a short half-life, they are effective sanitizing agents. Thus, as the air passes through the reactor 10, a large percentage (e.g., 80%-99.9%) of the airborne contaminants in the air received through the intake port are neutralized by the generated reactive oxygen species before the air is exhausted through an exhaust port, back into the environment to be treated. In this manner, the reactive oxygen species generated in the reactor 10 act as a sanitizer of the air passing through the reactor 10.

Ozone and hydrogen peroxide have a longer half life that other reactive oxygen species and, when produced, remain in the air and sanitize surfaces. In accordance with an exemplary aspect of the present invention, the apparatus eliminates the ozone, but trace amounts of vaporized hydrogen peroxide remain in the expelled air.

One of the reactive oxygen species generated by the reactor 10 is ozone (O₃). The generated ozone is introduced into the air in the reactor 10, and the ozone acts as a sanitizer of the air, surfaces, and environment. The ozone generated in the reactor 10 may be discharged with the air through the exhaust port. The ozone in the discharged air provides the beneficial preservative effects and acts as a sanitizer for any surfaces in the environment into which the air is discharged. Other reactive oxygen species, such as hydrogen peroxide, may also be discharged with the sanitized air and have sanitizing effects similar to ozone.

As indicated above, certain environments should remain ozone-free (e.g., 0 ppm ozone) or substantially ozone-free (e.g., equal to or less than approximately 0.02 ppm). Accordingly, the zero yield reactor of the present invention is capable of cancelling most or all of the ozone before the sanitized air is returned to the environment being treated.

The reactor 10 may include one or more reaction chambers 100 in which the reactive oxygen species are generated. The reaction chambers 100 may be arranged in an array within a housing (e.g., reactor tube) 102, as illustrated in FIG. 1. The housing 102 may include a round polyvinyl chloride (PVC) pipe of appropriate size. It is understood, however, that the housing may be of any desired shape or material. For example, the housing 102 may include the ductwork of an HVAC system or a refrigeration unit in a truck or walk-in cooler in a building.

The reaction chamber 100 may include a glass tube 106 lined with an inner stainless steel mesh 107 and wrapped in an outer stainless steel mesh 108. This configuration has been found to create a very effective corona that is able to generate a large amount of reactive oxygen species without using a static discharge and without producing material amounts of gases, such as nitrous oxide. While a round configuration for the reaction chamber is shown, the reaction chambers for generating reactive oxygen species may include different configurations and materials. For example, the reaction chambers may be formed of a glass tube 106 wrapped in stainless steel mesh with a copper tube coated with gold inside the glass tube at specific gaps. The reaction chambers may also be formed using appropriately configured plates of glass, ceramic or other materials with metal mesh on opposite sides. The particular configuration may be chosen to comport with the desired application of the zero yield reactor apparatus.

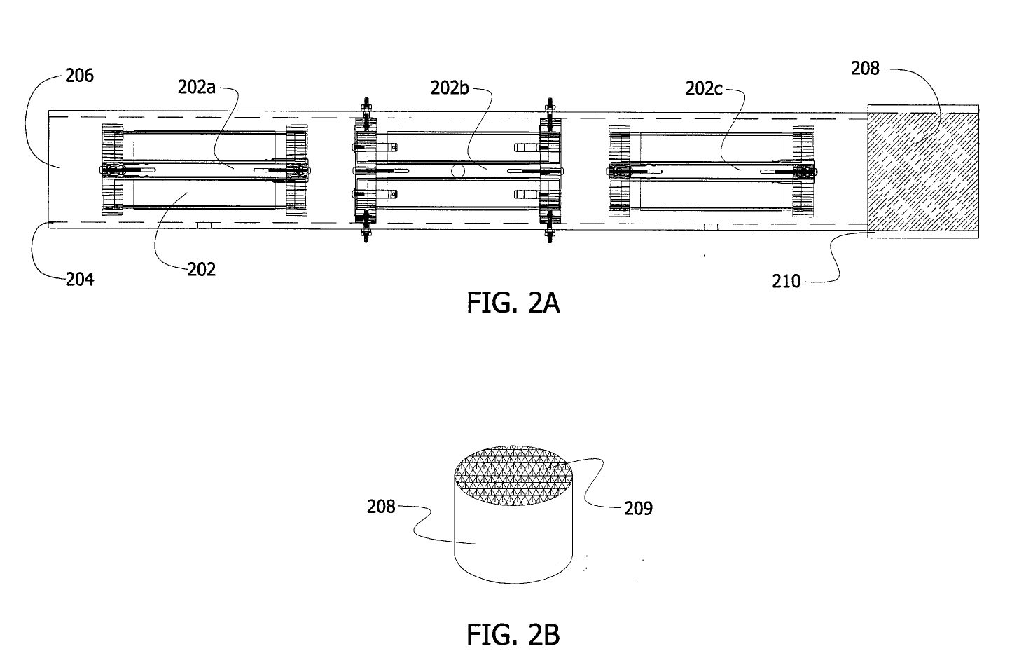

FIG. 2A illustrates a zero yield reactor 200 according to an exemplary, non-limiting embodiment of the present invention. The zero yield reactor 200 illustrated in FIG. 2 includes a plurality of arrays 202, as described above with respect to FIG. 1. Specifically, in the embodiment illustrated in FIG. 2, the zero yield reactor 200 includes three arrays 202a, 202b, and 202c, arranged in sequence inside of a reactor tube 204.

Incoming air is received from an environment to be sanitized through an intake port 206 of the reactor 200. Each of the arrays 202a-202c produces reactive oxygen species, including ozone, as described above.

The generated gas, including ozone and other reactive oxygen species, is passed through a filter (e.g., a manganese dioxide (MnO₂) filter), which catalytically converts the ozone back to oxygen gas. This process is exothermic and may produce enough heat to make the reaction go very quickly (e.g., 0.5-2 ns). Thus, the conversion process is a thermal-catalytic process. The process is a truly catalytic process, in that the catalyst is not consumed.

The manganese dioxide filter, illustrated in further detail in FIG. 2B, is disposed on an outtake port 210 of the reactor 200. The filter 208 includes a metal grid 209 that has been powder coated with manganese dioxide. The metal grid 209 is electrically charged with a current in a range of 0.005 VDC to 24 VDC (and more preferably at a current of 24 VDC).

The airflow to the reactor is set to approximately 54 CFM. The airflow of the reactor is set according to a size of the reactor. The larger the reactor, the faster the turbine can spin. This increased airflow compensates for the thermal-catalytic process taking place within the filter.

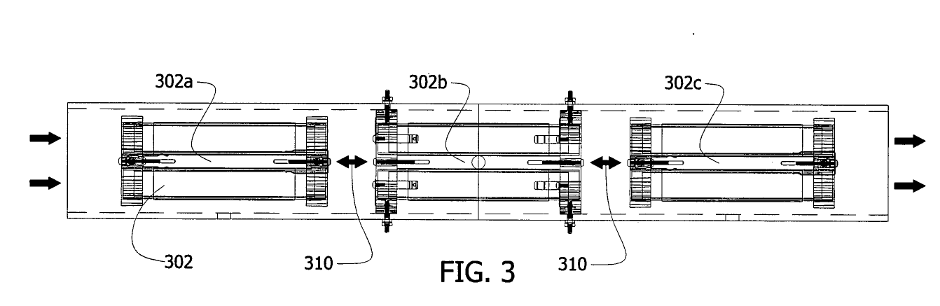

FIG. 3 illustrates a zero yield reactor 300 according to an exemplary, non-limiting embodiment of the present invention. The zero yield reactor 300 illustrated in FIG. 3 includes a plurality of arrays 302, as described above with respect to FIG. 1. Specifically, in the embodiment illustrated in FIG. 3, the zero yield reactor 300 includes three arrays 302a, 302b, and 302c, arranged in sequence inside of a reactor tube 304.

Each of the arrays 302 includes a four-cell array. The first array 302a is calibrated (set at 20 ppm) to produce the highest level of reactive oxygen species. The second array 302b is calibrated to produce a level of reactive oxygen species that is lower than that created in the first array 302a. The third array 302c is calibrated for the cancelation of ozone created by the first array 302a and the second array 302b. The first array 302a is calibrated to 0-12 kHz at 3500 VAC. The second array 302b is calibrated to 0-8 kHz at 2500 VAC. The third array 302c is calibrated to 12-20 kHz at 1500 VAC.

Each of the arrays 302 may be run at different power potentials and/or frequencies from one another. By running the arrays at different power potentials and/or frequencies, the reactor 300 is able to cancel (destruct) the production of ozone, while maintaining a steady production of other reactive oxygen species. Through the use of FET (field-effect transistor), frequencies reasonable for the creation of ozone are duplicated in order to cancel the production of ozone. The variables involved are based on the capacitance of the reactor (load).

For example, each of the arrays 302 may be run at the same power potential while the frequency for each array is varied. The first array 302a may be run at a frequency in a range of 0-12 kHz, the second array 302b may be run at a frequency in a range of 0-8 kHz, and the third array 302c may be run at a frequency in a range of 12-20 kHz.

Furthermore, the zero yield reactor 300 according to the present exemplary embodiment of the invention, may use a space cancellation differential 310 between each of the arrays. The space between the arrays acts as a buffer, allowing enough time for the reactive oxygen species to dissipate.

The zero yield reactor apparatus may be configured for general room sanitization applications where the zero yield reactor apparatus, or components thereof, may be placed in the ductwork of an HVAC system servicing the room to be sanitized. Alternatively, the zero yield reactor apparatus may be incorporated into the HVAC system of a facility to sanitize the air in the facility generally. Additionally, the zero yield reactor apparatus may be used to sanitize air to be introduced to a room from an outside source, as well as to treat exhaust air to remove odors and contaminants before releasing the air into the environment.

The zero yield reactor apparatus may be placed directly into a duct of an HVAC system so that some of the components are external to the duct in order to balance or reduce the weight of the zero yield reactor apparatus and create less stress on the ductwork. For example, one or more reactors 10 may be placed in the duct so that the air in the duct flows directly through the reactor 10 resulting in the generated reactive oxygen species sanitizing the air passing through, and the generated ozone cleaning the duct and being dispersed into the environment.

The level of ozone maintained in the environment into which the sanitized air containing ozone is dispersed, for example a room or building might vary from as low as 0.02 PPM to higher levels depending on regulations and safe operating conditions based on human presence. One skilled in the art will realize that the optimum level will be determined based on the size, configuration, and contents of the room. Further, one skilled in the art will recognize that the levels of ozone maintained in the environment used by people may be limited by governmental regulation. For example, OSHA regulations stipulate that eight hours of exposure to 0.1 PPM ozone is acceptable, and that fifteen minutes of exposure to 0.3 PPM ozone is acceptable. Use of higher concentrations may be dangerous. In the exemplary embodiment, the level of ozone will be controlled and maintained in accordance with governmental regulations. Higher levels of reactive oxygen species and ozone may be used during unoccupied periods for additional sanitation.

An additional feature of the zero yield reactor allows the cancellation mechanism to be turned on and off Accordingly, ozone may be used and distributed from the reactor during certain periods or under certain conditions. This can be accomplished with the use of a timer, sensor, or other mechanism that responds to user settings and environmental controls.

While the description refers to sanitizing air to be discharged into a room, space, or environment, it is to be understood that the invention can be applied to any defined environment. For example, an environment may be defined by solid surfaces or barriers, such as walls or product packaging, or defined by streams of forced gases, such as air screens or air curtains. Alternatively, the environment may be simply defined by the specific requirements of a desired application of the invention.

An exemplary application of the zero yield reactor apparatus would be for sanitizing sensitive areas of medical facilities, such as acute care areas and operating rooms. For example, the air circulation system of an operating room may include a network of ducts and vents that allow for the circulating of the air within the room without taking in air from outside the room. The apparatus, or elements thereof, may be placed in the duct work so that the air in the operating room may circulate through one or more reactors. It is to be understood that the apparatus may be employed in a wide variety of medical applications. For example, the sterilization of medical equipment storage cabinets and rooms, such as endoscope cabinets, and the sanitization of other rooms of medical facilities, such as waiting rooms, bathrooms, and food production areas.

As noted above, the apparatus may also be incorporated into the HVAC system of public buildings in order to treat the air within the buildings. In this manner, the apparatus may be used to sanitize the air and eliminate odors in the buildings. For example, office buildings, restaurants, malls, and the like would be particularly appropriate applications due to the large numbers of people that occupy the buildings and the need to sanitize the air in the buildings to provide a healthier, cleaner and more desirable environment for the occupants. The apparatus may further be employed to sanitize air that is to be exhausted out of buildings in order to eliminate or reduce contaminants and odors emitted from the building into the surrounding environment.

In another application of the invention, the sanitized air discharged into the environment may be directed through a nozzle or jet to permit directional control of the sanitized air. In this manner, the sanitized air can be actively directed to a specific location or area requiring the sanitizing effect of the discharged air. Similarly, the invention may be incorporated into a means for creating air curtains or air doors. For example, an air curtain can be created to substantially enclose a specified space in order to contain and control any undesirable odors or emissions from contents within the created space, or, alternatively, sanitize or preserve the contents within the created space.

The apparatus may further include a plurality of sensors and modules located within the apparatus and throughout the environment into which the sanitized air is discharged. The sensors and modules are used to measure pertinent variables, such as ozone levels, humidity, airflow, and temperature of the air in and around the apparatus. A programmable logic circuit (PLC) may be used to measure the performance of the apparatus based on data feedback from the plurality of sensors and modules. The PLC may store this information locally or report the information to a controller, which can be linked to the apparatus and to a central monitor and monitoring system, such as a computer or other dedicated device.

Furthermore, sensors that monitor dangerous contaminants in the air, such as biological weapons, could be used to control the units in case of detection in order to eliminate or contain a biological threat. Also, the apparatus of the present invention could be used in an air evacuation situation when air is drawn out of a contaminated space or building, and sanitizing that air to reduce or eliminate a threat outside of the space.

While the invention has been described in terms of several exemplary embodiments, those skilled in the art will recognize that the invention can be practiced with modification.

Further, it is noted that, Applicant's intent is to encompass equivalents of all claim elements, even if amended later during prosecution.

What is claimed is:

1. An apparatus for sanitizing air, comprising:
a generating unit that generates reactive oxygen species including ozone from oxygen in air, received by said generating unit, to be sanitized; and
a cancellation unit that cancels at least a portion of the ozone before the air is returned to an area to be sanitized.

2. The apparatus for sanitizing air according to claim 1, wherein an entirety of the ozone is canceled before the air is returned to the area to be sanitized.

3. The apparatus for sanitizing air according to claim 1, wherein an entirety of the ozone is canceled before the air is returned to the area to be sanitized and a continuous production of other reactive oxygen species is maintained.

4. The apparatus according to claim 1, wherein the reactive oxygen species comprise at least one of oxygen, atomic oxygen, superoxide, hydrogen peroxide, hydroxyl radical, and peroxynitrite.

5. An apparatus for sanitizing air, comprising:
a reactor comprising a plurality of arrays, said plurality of arrays comprising:
a reactive oxygen species generating array that generates reactive oxygen species including ozone from oxygen in air, received by said generating unit, to be sanitized; and
a cancellation array that cancels at least a portion of the ozone before the air is returned to an area to be sanitized.

6. The apparatus according to claim 5, wherein said plurality of arrays further comprises a reduction array, disposed between said reactive oxygen species generating array and said cancellation array, that produces a level of reactive oxygen species and ozone that is lower than a level produced by said reactive oxygen species generating array.

7. The apparatus according to claim 5, wherein the reactor comprises a space cancellation differential between individual arrays of said plurality of arrays.

8. The apparatus according to claim 5, wherein individual arrays of said plurality of arrays are operated at different power potentials.

9. The apparatus according to claim 5, wherein individual arrays of said plurality of arrays are operated at different frequencies.

10. The apparatus according to claim 5, wherein individual arrays of said plurality of arrays are operated at different power potentials and frequencies.

11. An apparatus for sanitizing air, comprising:
a reaction unit that generates reactive oxygen species including ozone from oxygen in air, received in the reaction unit, to be sanitized; and
a conversion filter that converts the ozone back to oxygen.

12. The apparatus according to claim 11, wherein said conversion filter comprises a catalytic filter.

13. The apparatus according to claim 11, wherein said filter comprises a catalyst, which is not consumed during conversion of the ozone.

14. The apparatus according to claim 11, wherein said conversion filter comprises manganese dioxide as a catalyst.

15. The apparatus according to claim 11, wherein said reaction unit comprises a plurality of arrays.

16. A method of sanitizing air, comprising:
generating reactive oxygen species including ozone from oxygen in air to be sanitized; and
canceling at least a portion of the ozone before the air is returned to an area to be sanitized.

17. The method according to claim 16, wherein an entirety of the ozone is canceled before the air is returned to the area to be sanitized and a continuous production of other reactive oxygen species is maintained.

18. The method according to claim 16, wherein the canceling at least a portion of the ozone before the air is returned to an area to be sanitized comprises applying a space cancellation differential.

19. The method according to claim 16, further comprising varying at least one of a power potential and a frequency at which a reactor is run.

20. The method according to claim 16, wherein the canceling at least a portion of the ozone before the air is returned to an area to be sanitized comprises converting the ozone back to oxygen.

21. The method according to claim 20, wherein said converting the ozone back to oxygen comprises passing the ozone through manganese dioxide.
